(19) United States
(12) Patent Application Publication
(10) Pub. No.: US 2022/0068434 A1
(43) Pub. Date: Mar. 3, 2022

(54) MONITORING MUTATIONS USING PRIOR KNOWLEDGE OF VARIANTS

(71) Applicant: Roche Sequencing Solutions, Inc., Pleasanton, CA (US)

(72) Inventors: Alexander Lovejoy, Newark, CA (US); Amrita Pati, San Ramon, CA (US); Daniel Klass, Lake Bluff, IL (US); Xiaoju Max Ma, San Carlos, CA (US); Weibing Shi, Brentwood, CA (US)

(21) Appl. No.: 17/346,060

(22) Filed: Jun. 11, 2021

Related U.S. Application Data
(63) Continuation of application No. PCT/EP2019/084893, filed on Dec. 12, 2019.
(60) Provisional application No. 62/778,725, filed on Dec. 12, 2018.

(51) Int. Cl. G16B 20/20; G16B 5/20; G16H 10/40; G16H 50/30; G16H 50/20

(52) U.S. Cl. CPC G16B 20/20; G16B 5/20; G16H 50/20; G16H 50/30; G16H 10/40

(57) ABSTRACT

Techniques for cancer patient management, and more particularly, to techniques for ultrasensitive detection of circulating nucleic acid with prior knowledge of variants to be monitored in the blood. An exemplary technique includes detecting one or more variants in a sample of cell free DNA from a subject. The one or more variants are selected from a plurality of variants known to be specific to a tumor or disease area of the subject. The technique further includes counting the detected one or more variants, determining a tumor burden based on the count of the one or more variants, and determining a statistical significance of the tumor burden based on whether the detection of the one or more variants is associate with true signals or background noise.

### REFERENCE TO PRIOR APPLICATIONS

The present application is a continuation of PCT Application No. PCT/EP2019/084893, filed on Dec. 12, 2019, which claims priority to U.S. Provisional Application No. 62/778725, filed on Dec. 12, 2018. all of which are incorporated by reference in their entireties.

### FIELD OF THE INVENTION

The present disclosure relates generally to cancer patient management, and more particularly, to techniques for ultrasensitive detection of circulating nucleic acid with prior knowledge of variants to be monitored in the blood.

### BACKGROUND

The development of plasma genotyping assays and other liquid biopsy assays has expanded the clinical utility of cell-free DNA (cfDNA) as a noninvasive cancer biomarker for cancer patient management. Plasma genotyping assays can noninvasively detect and quantify clinically relevant point mutations, insertions/deletions, amplifications, rearrangements, and aneuploidy within cfDNA. However, clinical use of cfDNA analysis requires exceedingly accurate assays for the genetic characterization of DNA fragments within the sample of interest. These assays must have high analytical sensitivity to detect clinically relevant genetic alterations from circulating tumor DNA (ctDNA) in a high background of wild-type DNA shed by nonmalignant cells. Plasma genotyping has potential clinical utility in early diagnosis, the detection of minimal residual disease (MRD), and the evaluation of treatment response and resistance.

MRD is the presence of residual tumor (e.g., small numbers of leukaemic cells) that remains in the patient during treatment or after treatment when the patient is in remission (i.e., no symptoms or signs of disease). A key technical challenge in cancer patient management is the detection of MRD. Recent studies have shown poor prognostic outcomes for patients even with ultra-low residual tumor burdens after treatment across a variety of cancer types based on the available technologies and algorithms Since changes in ctDNA levels correlate with relative changes in tumor burden, in order to detect MRD with an appropriate level of sensitivity, an assay must be able to detect ctDNA at least 30× lower than pre-treatment levels. However, clinical use of ctDNA analysis requires exceedingly accurate assays for the genetic characterization of DNA fragments within the sample of interest. These assays must have high analytical sensitivity to detect clinically relevant genetic alterations from ctDNA in a high background of wild-type DNA shed by nonmalignant cells. The difficulty inherent in achieving this limit of detection (LOD) is compounded by the small amount of cfDNA derived from a typical blood draw. Low allele frequencies or fractions ((AF), <0.5% mutant AF) are commonly seen in patients, particularly in the context of early detection or MRD. Detecting a single variant at sequencing depths commensurate with a typical genotyping test faces sensitivity limitations at low AFs, as the probability of observing a few or even a single molecule of a specific variant is drastically low. Accordingly, new techniques are desired for ultrasensitive detection of circulating nucleic acid are needed.

### BRIEF SUMMARY

Techniques are provided (e.g., a method, a system, non-transitory computer-readable medium storing code or instructions executable by one or more processors) for ultrasensitive detection of circulating nucleic acid with prior knowledge of variants to be monitored in the blood.

A system of one or more computers can be configured to perform particular operations or actions by virtue of having software, firmware, hardware, or a combination of them installed on the system that in operation causes or cause the system to perform the actions. One or more computer programs can be configured to perform particular operations or actions by virtue of including instructions that, when executed by data processing apparatus, cause the apparatus to perform the actions. One general aspect is directed to a method that includes: (a) obtaining, by a data processing system, sequence data for a plurality of target regions in a sample of cell free DNA from a subject, where the plurality of target regions are selected from a plurality of genomic regions that include a plurality of known variants, and the plurality of known variants are tagged with unique molecular identifiers. The method also includes (b) querying, by the data processing system, the sequence data for one or more variants of the plurality of known variants, and (c) calculating, by the data processing system, a first mutant molecule load (mml) for each of the queried one or more variants based on a first count of the unique molecular identifiers for each of the queried one or more variants. The method also includes (d) modeling, by the data processing system, background in the sample of cell free DNA, where the modeling includes randomly sampling the sequence data a predetermined number of times for one or more types of variants present in the plurality of genomic regions and calculating a second mml for each of the one or more variants in the random samples based on a second count of the unique molecular identifiers for each of the one or more variants in the random samples. The method also includes (e) comparing, by the data processing system, the second mml for each of the one or more variants in the random samples to the first mml for each of the queried one or more variants, and (f) generating, by the data processing system, a ratio based on the comparison, where the ratio is: (a number of the random samples where the second mml is greater than the first mml):(the predetermined number of times), and the ratio is a probability value for a null-hypothesis that the second mml of the background is greater than the first mml of the queried variant. The method also includes (g) determining, by the data processing system, a tumor burden for the subject based on the first mml for each of the queried one or more variants, and (h) determining, by the data processing system, a statistical significance of the tumor burden based on the probability value and a significance value. Other embodiments of this aspect include corresponding computer systems, apparatus, and computer programs recorded on one or more computer storage devices, each configured to perform the actions of the methods.

Implementations may include one or more of the following features. The method where the modeling may also include determining, by the data processing system, a distribution of the one or more types of variants present in the plurality of genomic regions, where the distribution includes a first type of variant and a second type of variant. The method where the randomly sampling the sequence data may include: (i) selecting at least one base associated with the first type of variant a predetermined number of times from the sequence data and counting a number of molecules supporting the first type of variant, and (ii) selecting at least one base associated with the second type of variant a predetermined number of times from the sequence data and counting a number of molecules supporting the second type of variant. The method where the at least one base associated with the first type of variant may be selected based on a multinucleotide context of the first type of variant, and the at least one base associated with the second type of variant may be selected based on the multinucleotide context of the second type of variant.

Implementations may include one or more of the following features. The method may also include determining, by the data processing system, the significance value as a variable significance value based on a number of the queried one or more variants while maintaining a predetermined false discovery rate. The method where the determining the variable significance value may include: (i) determining a different significance value for each number of the one or more variants of the plurality of variants that are capable of being queried while maintaining the predetermined false discovery rate, and selecting the significance value for the number of the queried one or more variants from the determined different significance values; or (ii) determining an equation relating the significance value to the number of the queried one or more variants while maintaining a predetermined false discovery rate. The method where the significance value may be determined based on the number of the queried one or more variants that are associated with a subject tumor.

Implementations may include one or more of the following features. The method may also include determining, by the data processing system, whether the subject has minimal residual disease based on the statistical significance of the tumor burden. The method may also include: predicting, by the data processing system, a clinical outcome of a treatment regimen for the subject based upon whether the subject has the minimal residual disease, and upon determining the subject does have minimal residual disease and predicting a negative clinical outcome, modifying the treatment regimen of the subject. The method where the one or more variants may be detected using a diagnostic assay including probes specific to the plurality of known variants.

One general aspect directed to a method that includes: (a) obtaining, by a data processing system, sequence data for a plurality of target regions in a sample of cell free DNA from a subject, where the plurality of target regions are selected from a plurality of genomic regions that include a plurality of known variants. The method also includes (b) querying, by the data processing system, the sequence data for one or more variants of the plurality of known variants, and (c) calculating, by the data processing system, a first number of mutant molecules for each of the queried one or more variants based on an allele fraction for each of the queried one or more variants. The method also includes (d) modeling, by the data processing system, background in the sample of cell free DNA, where the modeling includes randomly sampling the sequence data a predetermined number of times for one or more types of variants present in the plurality of genomic regions and calculating a second number of mutant molecules for each of the one or more variants in the random samples based on an allele fraction for each of the one or more variants in the random samples. The method also includes (e) comparing, by the data processing system, the second number of mutant molecules for each of the one or more variants in the random samples to the first number of mutant molecules for each of the queried one or more variants, and (f) generating, by the data processing system, a ratio based on the comparison, where the ratio is: (a number of the random samples where the second number of mutant molecules is greater than the first number of mutant molecules):(the predetermined number of times), and the ratio is a probability value for a null-hypothesis that the second number of mutant molecules of the background is greater than the first number of mutant molecules of the queried variant. The method also includes (g) determining, by the data processing system, a variable significance value based on a number of the queried one or more variants while maintaining a predetermined false discovery rate, where the determining the variable significance value includes: (i) determining a different significance value for each number of the one or more variants of the plurality of known variants that are capable of being queried while maintaining the predetermined false discovery rate, and selecting the significance value for the number of the queried one or more variants from the determined different significance values; or (ii) determining an equation relating the significance value to the number of the queried one or more variants while maintaining a predetermined false discovery rate. The method also includes (h) determining, by the data processing system, a tumor burden for the subject based on the first number of mutant molecules for each of the queried one or more variants, and (i) determining, by the data processing system, a statistical significance of the tumor burden based on the probability value and the variable significance value. Other embodiments of this aspect include corresponding computer systems, apparatus, and computer programs recorded on one or more computer storage devices, each configured to perform the actions of the methods.

Implementations may include one or more of the following features. The method where the modeling may also include determining, by the data processing system, a distribution of the one or more types of variants present in the plurality of genomic regions, where the distribution includes a first type of variant and a second type of variant. The method where the randomly sampling the sequence data may include: (i) selecting at least one base associated with the first type of variant a predetermined number of times from the sequence data and quantifying a number of molecules supporting the first type of variant, and (ii) selecting at least one base associated with the second type of variant a predetermined number of times from the sequence data and quantifying a number of molecules supporting the second type of variant. The method where the at least one base associated with the first type of variant may be selected based on a multinucleotide context of the first type of variant, and the at least one base associated with the second type of variant may be selected based on the multinucleotide context of the second type of variant.

Implementations may include one or more of the following features. The method where the variable significance value may be determined based on the number of the queried one or more variants that are associated with a subject tumor. The method may also include determining, by the data processing system, whether the subject has minimal residual disease based on the statistical significance of the tumor burden. The method may also include: predicting, by the data processing system, a clinical outcome of a treatment regimen for the subject based upon whether the subject has the minimal residual disease, and upon determining the subject does have minimal residual disease and predicting a negative clinical outcome, modifying the treatment regimen of the subject.

One general aspect is directed to a method of diagnosing a patient with minimal residual disease, where the method includes: (a) detecting, by a data processing system, one or more variants in a sample of cell free DNA from a subject, where the one or more variants are selected from a plurality of variants known to be specific to a tumor or disease area of the subject. The method of diagnosing also includes (b) counting, by the data processing system, the detected one or more variants, and (c) determining, by the data processing system, a tumor burden based on the count of the one or more variants. The method of diagnosing also includes (d) determining, by the data processing system, a statistical significance of the tumor burden, where the determining the statistical significance includes: (i) modeling background noise using a multinucleotide context of base changes from randomly selected positions in sequence data for the sample of cell free DNA to define an empirically derived probability value, (ii) determining a variable significance value based on a number of the detected one or more variants while maintaining a predetermined false discovery rate, and (iii) comparing the probability value to the variable significance value, where the lower the probability value compared to the variable significance value, the higher the statistical significance of the tumor burden. The method of diagnosing also includes (e) determining, by the data processing system, whether the subject has minimal residual disease based on the statistical significance of the tumor burden. Other embodiments of this aspect include corresponding computer systems, apparatus, and computer programs recorded on one or more computer storage devices, each configured to perform the actions of the methods.

Implementations may include one or more of the following features. The method where when the tumor burden is greater than a threshold value and the probability value is less than the significance value, determining that the subject has minimal residual disease. The method where the threshold value is a predetermined cutoff of a tumor burden value that is associated with a presence of the minimal residual disease in the subject. The method where when the tumor burden is greater than the threshold value and the probability value is greater than the significance value, determining that the subject does not have minimal residual disease. The method where when the tumor burden is less than the threshold value and the probability value is less than the significance value, determining that the subject does not have minimal residual disease. The method where when the tumor burden is less than the threshold value and the probability value is greater than the significance value, determining that the subject does not have minimal residual disease. The method may further include: predicting, by the data processing system, a clinical outcome of a treatment regimen for the subject based upon whether the subject has the minimal residual disease. The method may also include upon determining the subject does have minimal residual disease and predicting a negative clinical outcome, modifying the treatment regimen of the subject.

One general aspect is directed to a method of performing a survivability analysis, where the method includes: (a) detecting, by a data processing system, one or more variants in a sample of cell free DNA from a subject, where the one or more variants are selected from a plurality of variants known to be specific to a tumor or disease area of the subject. The method of analysis also includes (b) counting, by the data processing system, the detected one or more variants, and (c) determining, by the data processing system, a statistical significance of the count of the one or more variants, where the determining the statistical significance includes: (i) modeling background noise using a multinucleotide context of base changes from randomly selected positions in sequence data for the sample of cell free DNA to define an empirically derived probability value, (ii) determining a variable significance value based on a number of the detected one or more variants while maintaining a predetermined false discovery rate, and (iii) comparing the probability value to the variable significance value, where the lower the probability value compared to the variable significance value, the higher the statistical significance of the count of the one or more variants. The method of analysis also includes (d) repeating steps (a)-(c) for a predetermined number of samples of cell free DNA from the subject at various points prior to and during a treatment regimen to obtain the count of the one or more variants for each of the samples of cell free DNA and the statistical significance associated with each of the counts of the one or more variants. The method of analysis also includes (e) predicting, by the data processing system, a clinical outcome of the treatment regimen for the subject based upon the count of the one or more variants for each of the samples of cell free DNA and the statistical significance associated with each of the counts of the one or more variants. Other embodiments of this aspect include corresponding computer systems, apparatus, and computer programs recorded on one or more computer storage devices, each configured to perform the actions of the methods.

Implementations may include one or more of the following features. The method where the predicting the clinical outcome of the treatment regimen may include: analyzing, using a continuous responder algorithm, the count of the one or more variants for each of the samples of cell free DNA and the statistical significance associated with each of the counts of the one or more variant to classify the subject as a responder or a non-responder. The method where when the subject is classified as the responder, the clinical outcome of the treatment regimen for the subject may be predicted to be a positive clinical outcome; and when the subject is classified as the non-responder, the clinical outcome of the treatment regimen for the subject may be predicted to be a negative clinical outcome.

The techniques described above and below may be implemented in a number of ways and in a number of contexts. Several example implementations and contexts are provided with reference to the following figures, as described below in more detail. However, the following implementations and contexts are but a few of many.

### BRIEF DESCRIPTION OF THE DRAWINGS

FIG. 1 depicts a flowchart illustrating a process for ultrasensitive detection of circulating nucleic acid with prior knowledge of variants to be monitored in the blood in accordance with various embodiments.

FIG. 2 depicts a block diagram of a sequence analytical system in accordance with various embodiments.

FIG. 3 depicts a block diagram of a computing system or data processing system in accordance with various embodiments.

FIGS. 4A and 4B depict monitoring sensitivity at 0.002%, 0.004% and 0.01% using mean allele frequencies or fractions (AF) and mutant molecule load (MML) in accordance with various embodiments.

FIGS. 5A and 5B depict a comparison of sensitivity for 5% false discovery rate using trinucleotides (y-axis) vs. single nucleotides (x-axis) for two spikes in accordance with various embodiments.

FIGS. 6A-6D depict p-value cutoffs for consistent 5% false discovery rate or 95% specificity plotted against number of reporters in accordance with various embodiments.

FIG. 7 depicts a difference in p-value thresholds for 5% false discovery rate between non-small cell lung cancer (NSCLC) and colorectal cancer (CRC) in accordance with various embodiments.

FIG. 8 depicts a spike mixture experiment design for sensitivity experiments in accordance with various embodiments.

FIG. 9 depicts a flowchart illustrating a process for diagnosing a patient with minimal residual disease in accordance with various embodiments.

FIG. 10 depicts a flowchart illustrating a process for performing a survivability analysis in accordance with various embodiments.

### DETAILED DESCRIPTION

In the following description, various embodiments will be described. For purposes of explanation, specific configurations and details are set forth in order to provide a thorough understanding of the embodiments. However, it will also be apparent to one skilled in the art that the embodiments may be practiced without the specific details. Furthermore, well-known features may be omitted or simplified in order not to obscure the embodiment being described.

### I. Introduction

In various embodiments, techniques are provided (e.g., a method, a system, non-transitory computer-readable medium storing code or instructions executable by one or more processors) for ultrasensitive detection of circulating nucleic acid with prior knowledge of variants to be monitored in the blood. In some embodiments, the circulating nucleic acid is ctDNA, which originates directly from the tumor or from circulating tumor cells (CTCs), which are viable, intact tumor cells that shed from primary tumors and enter the bloodstream or lymphatic system. ctDNA is different from cfDNA, which is a broader term that describes DNA that is freely circulating in the bloodstream, but is not necessarily of tumor origin. Because ctDNA may reflect the entire tumor genome, it has gained traction for its potential clinical utility. For example, liquid biopsies of ctDNA may be obtained in a noninvasive form such as blood draws at various time points to monitor tumor progression throughout the treatment regimen.

Variants specific to a subject's tumor are conventionally identified using DNA derived from a pre-treatment tissue or plasma sample. Following that, the variants (also known as “reporters”) are queried in cfDNA derived from later samples from the same subject. Because these multiple reporters are known to be specific to the tumor, detection of one or more of these reporters can be sufficient evidence for ctDNA detection. This means, that, even if the ctDNA is present below the lower limit of detection for a given reporter, random sampling of multiple independent reporters can allow detection of the presence of ctDNA in a sample to a higher degree of sensitivity.

A significant aspect to confidently calling the presence of ctDNA using this monitoring technique, and alternative forms thereof, is the ability to differentiate true signal from background noise. In order to do this, conventional methods use a Monte Carlo algorithm to model the background noise in a given sample. In the conventional methods, base changes that correspond to the base changes in the true reporter list to be monitored are randomly selected from sequence data from the cfDNA. These trials are repeated a number of time (e.g., 10,000 times) and each time, the mean variant allele fraction (mean AF) of the background of the sample is compared with the mean AF derived from the true reporter list. A ratio is generated between the number of trials, where the observed mean AF is greater than the mean AF derived from the true reporter list indicating the random probability of background noise contributing to high mean AF signals, and the number of total trials (e.g., 10, 000 trials). This ratio can be interpreted as a p-value for the null hypothesis that the background generated mean AF is greater than the true-reporter list derived mean AF since it measures the random probability of success for the null hypothesis. The lower the p-value, the higher the statistical significance associated with the tumor burden computed from the true reporter list.

A problem associated with confidently determining the presence of ctDNA and predicting the statistical significance associated with a tumor burden derived from the presence of the ctDNA is less than optimal sensitivity and specificity in the conventional monitoring techniques. Sensitivity and specificity are statistical measures of the performance of a binary classification test, also known in statistics as a classification function. Sensitivity (also called the true positive rate, the recall, or probability of detection in some fields) measures the proportion of actual positives that are correctly identified as such (e.g., the percentage of sick people who are correctly identified as having the condition). Specificity (also called the true negative rate) measures the proportion of actual negatives that are correctly identified as such (e.g., the percentage of healthy people who are correctly identified as not having the condition). A perfect predictor would be described as 100% sensitive, meaning all sick individuals are correctly identified as sick, and 100% specific, meaning no healthy individuals are incorrectly identified as sick. In reality, any non-deterministic predictor such as calling the presence of ctDNA will possess a minimum error known as the Bayes error rate. The conventional monitoring techniques, however, do not come close to optimal sensitivity and specificity (i.e., approaching Bayes error rate).

To address these problems, various embodiments disclosed herein are directed to systems and methods that implement one or more of the following techniques: (i) quantification of ctDNA in a sample using mutant molecules as defined by the sequencing assay, (ii) use of multinucleotide context of base changes from randomly selected positions to define the empirically derived p-value, and (iii) a technique to ensure specificity levels in a data derived manner using data-driven p-value threshold selection. For example, one illustrative embodiment of the present disclosure comprises: (a) detecting, by a data processing system, one or more variants in a sample of cell free DNA from a subject, wherein the one or more variants are selected from a plurality of variants known to be specific to a tumor or disease area of the subject; (b) counting, by the data processing system, the detected one or more variants; (c) determining, by the data processing system, a tumor burden based on the count of the one or more variants; and (d) determining, by the data processing system, a statistical significance of the tumor burden based on whether the detection of the one or more variants is associate with true signals or background noise. The determining the statistical significance may comprise one or more of the following: (i) modeling background noise using a multinucleotide context of base changes from randomly selected positions in sequence data for the sample of cell free DNA to define an empirically derived probability value, (ii) determining a variable significance value based on a number of the detected one or more variants while maintaining a predetermined false discovery rate, and (iii) comparing the probability value to the variable significance value, where the lower the probability value compared to the variable significance value, the higher the statistical significance of the tumor burden. As used herein, when an action is “triggered by” or “based on” something, this means the action is triggered or based at least in part on at least a part of the something.

Optionally, the techniques further include: (a) determining, by the data processing system, whether the subject has minimal residual disease based on the statistical significance of the tumor burden; (b) predicting, by the data processing system, a clinical outcome of a treatment regimen for the subject based upon whether the subject has the minimal residual disease; and (c) upon determining the subject does have minimal residual disease and predicting a negative clinical outcome, modifying the treatment regimen of the subject.

An alternative illustrative embodiment of the present disclosure comprises:
(a) detecting, by a data processing system, one or more variants in a sample of cell free DNA from a subject, wherein the one or more variants are selected from a plurality of variants known to be specific to a tumor or disease area of the subject;
(b) counting, by the data processing system, the detected one or more variants; and
(c) determining, by the data processing system, a statistical significance of the count of the one or more variants based on whether the detection of the one or more variants is associate with true signals or background noise. The determining the statistical significance may comprise one or more of the following: (i) modeling background noise using a multinucleotide context of base changes from randomly selected positions in sequence data for the sample of cell free DNA to define an empirically derived probability value, (ii) determining a variable significance value based on a number of the detected one or more variants while maintaining a predetermined false discovery rate, and (iii) comparing the probability value to the variable significance value, where the lower the probability value compared to the variable significance value, the higher the statistical significance of the count of the one or more variants

Optionally, the alternative techniques further include: (a) predicting, by the data processing system, a clinical outcome of the treatment regimen for the subject by analyzing, using a continuous responder algorithm, the count of the one or more variants for each of the samples of cell free DNA and the statistical significance associated with each of the counts of the one or more variant to classify the subject as a responder or a non-responder; (b) when the subject is classified as the responder, the clinical outcome of the treatment regimen for the subject is predicted to be a positive clinical outcome; and when the subject is classified as the non-responder, the clinical outcome of the treatment regimen for the subject is predicted to be a negative clinical outcome.

Advantageously, these approaches improve upon determining the presence of ctDNA in a sample. The improved confidence in determining the presence of ctDNA can then be used in downstream analysis to predict with greater sensitivity and specificity the statistical significance associated with a tumor burden or a count of one or more variants derived from the presence of the ctDNA.

### II. Techniques for Ultrasensitive Detection of Circulating Nucleic Acid

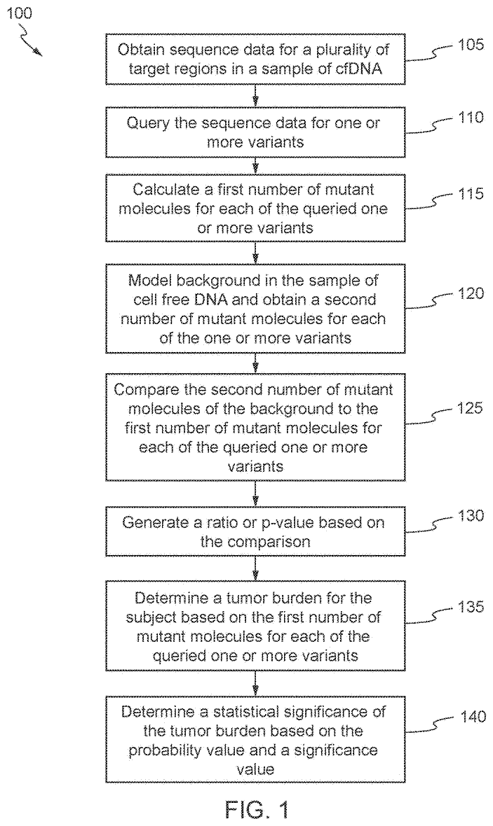

FIG. 1 illustrates processes and operations for ultrasensitive detection of circulating nucleic acid with prior knowledge of variants to be monitored in the blood. Individual embodiments may be described as a process which is depicted as a flowchart, a flow diagram, a data flow diagram, a structure diagram, or a block diagram. Although a flowchart may describe the operations as a sequential process, many of the operations may be performed in parallel or concurrently. In addition, the order of the operations may be re-arranged. A process is terminated when its operations are completed, but could have additional steps not included in a figure or the description thereof. A process may correspond to a method, a function, a procedure, a subroutine, a subprogram, etc. When a process corresponds to a function, its termination may correspond to a return of the function to the calling function or the main function.

The processes and/or operations depicted in FIG. 1 may be implemented in software (e.g., code, instructions, program) executed by one or more processing units (e.g., processors cores), hardware, or combinations thereof. The software may be stored in a memory (e.g., on a memory device, on a non-transitory computer-readable storage medium). The particular series of processing steps in FIG. 1 is not intended to be limiting. Other sequences of steps may also be performed according to alternative embodiments. For example, in alternative embodiments the steps outlined herein may be performed in a different order. Moreover, the individual steps illustrated in FIG. 1 may include multiple sub-steps that may be performed in various sequences as appropriate to the individual step. Furthermore, operations or steps may be added or removed depending on the particular applications. One of ordinary skill in the art would recognize many variations, modifications, and alternatives.

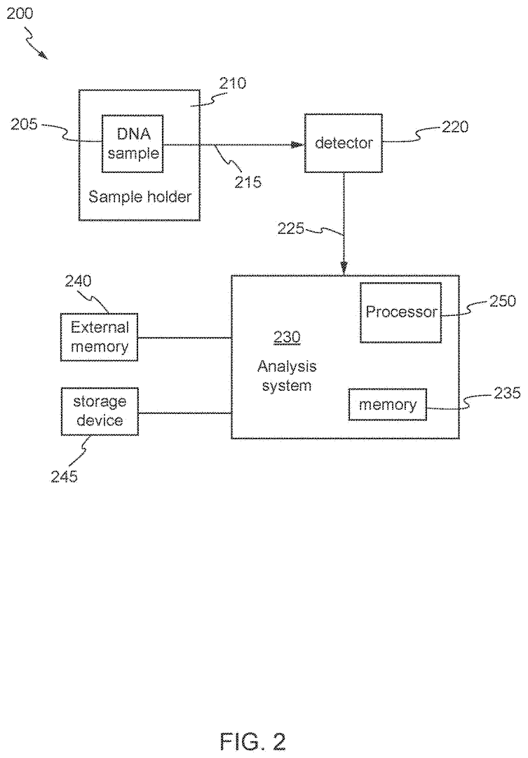

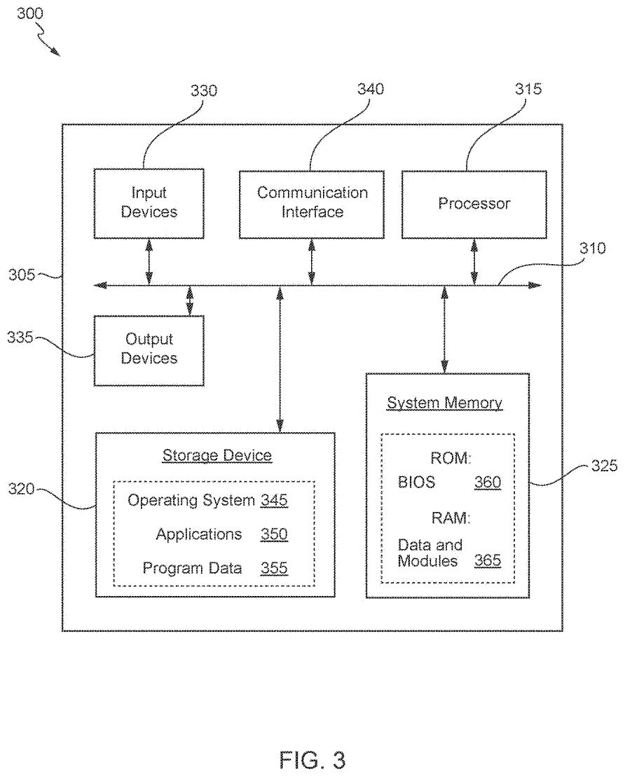

FIG. 1 shows a flowchart 100 that illustrates a process for ultrasensitive detection of circulating nucleic acid with prior knowledge of variants to be monitored in the blood. In some embodiments, the processes depicted in flowchart 100 may be implemented by the architecture, systems, and techniques depicted in FIGS. 2 and 3. At step 105, sequence data is obtained for a plurality of target regions in a sample of cfDNA from a subject (e.g., a patient). The sequence data includes an inferred plurality of nucleotide sequences of the cfDNA known as reads. The sequence data may be obtained and analyzed using any suitable sequencing technique, as described in detail in Section III. In some embodiments, one or more samples having cfDNA are obtained (e.g., by drawing blood from a subject), sequenced, by a sequence analytical system, to generate sequence data for the cfDNA, and the sequence data is analyzed, by a data processing system, to provide some output such as tumor burden and a statistical significance of a tumor burden. In other embodiments, the sequence data is obtained, by the data processing system, from any source (public or private) in a suitable manner, and analyzed, by the data processing system, to provide some output such as tumor burden and a statistical significance of a tumor burden.

The plurality of target regions may be selected from a plurality of genomic regions that comprise a plurality of known variants. In some embodiments, the plurality of genomic regions are known to be recurrently mutated in the subject's tumor. For example, a list of prior known variants specific to the subject's tumor may be obtained, and the plurality of target regions may be selected from a library of recurrently mutated genomic regions associated with the known variants. The list of prior known variants may be generated by identifying known driver genes, i.e., genes that are known to be mutated frequently in the subject's tumor, and selecting variants within the identified driver genes that are specific to the tumor of the subject, i.e., variants previously identified in tumor DNA, ctDNA, or CTC DNA derived from a pre-treatment tissue or plasma sample from the subject. In other embodiments, the plurality of genomic regions are known to be recurrently mutated in a population having a tumor similar to the subject's tumor. For example, a list of prior known variants specific to a tumor or disease area similar to the subject's tumor or disease area may be obtained, and the plurality of target regions may be selected from a library of recurrently mutated genomic regions associated with the known variants. The list of prior know variants may be generated by identifying known driver genes, i.e., genes that are known to be mutated frequently in the particular cancer effecting the subject, selecting genomic regions in the driver genes that contain recurrent mutations in multiple subjects with the particular cancer, and ranking those selections to maximize the possibility of identifying variants in those regions in the sample of cfDNA.

As used herein, a “mutation” is the changing of the structure of a gene, resulting in a variant form that may be transmitted to subsequent generations, caused by the alteration of single base units in DNA, or the deletion, insertion, or rearrangement of larger sections of genes or chromosomes. A recurrent mutation is a mutation that has been identified multiple times in an individual or in more than one individual. As used herein, a “variant” or “variant form” is a form or version of a gene or sequence of nucleotides that. differs in some respect from other forms of the same gene or sequence of nucleotides, or from a reference gene or sequence. Examples of variants include: (i) single nucleotide polymorphisms (SNPs), which are DNA sequence variations that occur when a single nucleotide differs from the reference sequence, (ii) insertions, which occur when additional nucleotides are inserted in a sequence, relative to the reference sequence, (iii) deletions, which occur when there are missing nucleotides, relative to the reference sequence, (iv) substitutions which occur when multiple nucleotides are altered from the reference sequence, and (v) structural variants, which are changes where large sections of a chromosome or even whole chromosomes are duplicated, deleted or rearranged in some manner. Mutations in a gene or gene product can be detected in tumors or other body samples such as urine, spinal fluid, sputum, whole blood, or blood serum. For example, ctDNA, which originates directly from the tumor or from CTCs may appear in such body samples. In accordance with various embodiments, the nucleic acid detection methods discussed herein are capable of detecting mutant cells and ctDNA in a background of non-tumor cells in a wide variety of sample types including urine, spinal fluid, sputum, whole blood, or blood serum.

In some embodiments, the plurality of variants are tagged with unique molecular identifiers. An identifier can be any suitable detectable label incorporated into or attached to a nucleic acid (e.g., a polynucleotide) that allows detection and/or identification of nucleic acids that comprise the identifier. In some embodiments, an identifier is incorporated into or attached to a nucleic acid during a sequencing method (e.g., by a polymerase). Non-limiting examples of identifiers include nucleic acid tags, nucleic acid indexes or barcodes, a radiolabel (e.g., an isotope), metallic label, a fluorescent label, a chemiluminescent label, a phosphorescent label, a fluorophore quencher, a dye, a protein (e.g., an enzyme, an antibody or part thereof, a linker, a member of a binding pair), the like or combinations thereof. In some embodiments, an identifier (e.g., a nucleic acid index or barcode) is a unique, known and/or identifiable sequence of nucleotides or nucleotide analogues added to each sequence read to distinguish individual nucleotide molecules.

At step 110, the sequence data is queried for one or more variants of the plurality of variants. In some embodiments, the query includes searching the sequence data for evidence of the plurality of variants (reporters) and calling or identifying one or more variants of the plurality of variants in the sequence data. The sequence data may be queried using any suitable variant calling technique. For example, techniques may include the alignment of short reads and application of various statistical algorithms and models such as Bayesian models Probabilistic models, Fisher's Exact Test, and string graphs to call or identify the one or more variants in the sequence data. These algorithms and models specialize in calling or identifying discordant sequences in the ever-changing heterogeneous somatic cells in tumors, and are known generally as somatic variant callers. The algorithms and models use various parameters to identify low-frequency mutations in ctDNA compared to relatively normal cfDNA. In some embodiments, a deep read depth >1000× is preferred to discern true variants from artifacts when a probabilistic model is implemented.

At step 115, a first number of mutant molecules for each of the queried one or more variants is calculated. In some embodiments, the first number of mutant molecules for each of the queried one or more variants is calculated based on an allele frequency or allele fraction for each of the queried one or more variants. The allele frequency is the fraction of all the occurrences of a given allele or variant and the total number of target region copies with the cfDNA sample. For example, either a mean allele fraction of the queried one or more variants in the cfDNA or a mass of the cfDNA per mL of plasma combined with the mean allele fraction of the queried one or more variants in the cfDNA is used to calculate the first number of mutant molecules (or mutant copies of the genome) per mL of plasma for each of the queried one or more variants. Allele frequencies or allele fractions suitable for use with various embodiments described herein, may be calculated using known techniques.

In other embodiments, the first number of mutant molecules is a first mutant molecule load (MML) for each of the queried one or more variants, and the first mutant molecule load (MML) is calculated based on a first count of the unique molecular identifiers for each of the queried one or more variants. For example, the sequence assay used to obtain the sequence data may include unique molecule identifiers to distinguish individual nucleotide molecules, and thus, the number of sequenced molecules that contained the queried one or more variants can be counted using the unique molecule identifiers, as described in detail in Section IV. Detection and quantification of the unique molecule identifiers can be performed by any suitable technique, non-limiting examples of which include flow cytometry, quantitative polymerase chain reaction (qPCR), gel electrophoresis, a luminometer, a fluorometer, a spectrophotometer, a suitable gene-chip or microarray analysis, Western blot, mass spectrometry, chromatography, cytofluorimetric analysis, fluorescence microscopy, a suitable fluorescence or digital imaging method, confocal laser scanning microscopy, laser scanning cytometry, affinity chromatography, manual batch mode separation, electric field suspension, a suitable nucleic acid sequencing method and/or nucleic acid sequencing apparatus, the like and combinations thereof.

At step 120, background in the sample of cell free DNA is modeled. The modeling comprises randomly sampling the sequence data a predetermined number of times for one or more types of variants present in the plurality of genomic regions and calculating a second number of mutant molecules for each of the one or more variants in the random samples. In some embodiments, the second number of mutant molecules for each of the one or more variants in the random samples is calculated based on an allele fraction for each of the one or more variants in the random samples. In other embodiments, the second number of mutant molecules is a second MML for each of the one or more variants in the random samples, and the second MML for each of the one or more variants in the random samples is calculated based on a second count of the unique molecular identifiers for each of the one or more variants in the random samples.

In various embodiments, modeling the background includes determining a distribution of the one or more types of variants present in the plurality of genomic regions. The one or more types of variants may include SNP types (e.g., A>C, A>T, and G>A), insertion types, deletion types, substitution types, and structural variant types. In certain embodiments, the distribution comprises a first type of variant and a second type of variant. The random sampling of the sequence data for the first type of variant and the second type of variant may comprise: (i) selecting at least one base associated with the first type of variant a predetermined number of times from the sequence data and counting a number of molecules supporting the first type of variant, and (ii) selecting at least one base associated with the second type of variant a predetermined number of times from the sequence data and counting a number of molecules supporting the second type of variant. Alternatively, the random sampling of the sequence data for the first type of variant and the second type of variant may comprise: (i) selecting two or more bases associated with the first type of variant a predetermined number of times from the sequence data and counting a number of molecules supporting the first type of variant, and (ii) selecting two or more bases associated with the second type of variant a predetermined number of times from the sequence data and counting a number of molecules supporting the second type of variant. In some embodiments, the predetermined number of times the base(s) is selected for the first type of variant is identical to the predetermined number of times the base(s) is selected for the second type of variant for each sampling. In some embodiments, the base(s) associated with the first type of variant is selected based on a multinucleotide context of the first type of variant (e.g., dinucleotide, tetranucleotide, pentanucleotide, or other contexts), and the base(s) associated with the second type of variant is selected based on the multinucleotide context of the second type of variant (e.g., dinucleotide, tetranucleotide, pentanucleotide, or other contexts), as described in detail in Section V.

At step 125, the second number of mutant molecules of the background for each of the one or more variants in the random samples is compared to the first number of mutant molecules for each of the queried one or more variants. At step 130, a ratio is generated based on the comparison. In some embodiments, the ratio is: (a number of the random samples where the second number of mutant molecules of the background is greater than the first number of mutant molecules of the queried variant):(the predetermined number of times). Thus, the ratio is a probability value (p-value) for a null-hypothesis that the second number of mutant molecules of the background is greater than the first number of mutant molecules of the queried variant. For example, a Monte Carlo algorithm may be used to model the background where base changes that correspond to the base changes in the list of prior known variants to be monitored (i.e., the number of one or more type of variants present in the plurality of genomic regions) are randomly selected a number of times (e.g., one time) from the sequencing data from the sample of cfDNA and a number of molecules supporting the base changes are calculated to obtain a second number of mutant molecules of the background for each of the one or more variants in the random samples. These trials are repeated a predetermined number of times (e.g., 10,000 times) and each time, the second number of mutant molecules of the background is compared with the first number of mutant molecules for each of the queried one or more variants derived from the list of prior known variants to be monitored. A ratio is generated between the number of trials, where the observed second number of mutant molecules of the background is greater than the first number of mutant molecules of the queried variant indicating the random probability of background noise contributing to high mutant molecule signals, and the predetermined number of trials. This ratio may be interpreted as a p-value for the null hypothesis that the background generated second number of mutant molecules is greater than the reporter generated first number of mutant molecules since the ratio measures the random probability of success for the null hypothesis. The lower the p-value, the higher the statistical significance associated with the tumor burden computed from the list of prior known variants (reporter list).

At step 135, a tumor burden for the subject is determined based on the first number of mutant molecules for each of the queried one or more variants. As used herein, a “tumor burden” is a quantitation of the amount of tumor-derived DNA, either calculated by MMPM or by mean AF of tumor variants. In some embodiments, the quantitation of the amount of tumor-derived DNA is proportional to a systemic tumor burden for the subject. At step 140, a statistical significance of the tumor burden is determined based on the probability value and a significance value. In statistical hypothesis testing, a result has statistical significance when it is very unlikely to have occurred given the null hypothesis. As discussed with respect to step 130, the ratio (a number of the random samples where the second number of mutant molecules of the background is greater than the first number of mutant molecules of the queried variant):(the predetermined number of times) is the probability value (p-value) for the null-hypothesis that the second number of mutant molecules of the background is greater than the first number of mutant molecules of the queried variant. The null hypothesis may be rejected if the p-value is less than a predetermined level, which is called the significance level, and is the probability of rejecting the null hypothesis given that it is true (a type I error). The significance level is typically set at or below 5%. For example, a p-value cutoff of 0.05 corresponds to the probability of roughly 5% of trials having a mean AF greater than the mean AF derived from the reporter list, or a false discovery rate (FDR) of 5%. Alternatively, it has been discovered that the number of reporters can play a role in determining the exact p-value cutoff for a 5% FDR and the disease area in somatic oncology has implications as well. In various embodiments, techniques are provided for the determination of a variable p-value cutoff or a variable significance value based on the number of reporters queried in step 110 and/or the disease area of the subject (e.g., a tumor present in the subject), which allows for the detection of ctDNA-positivity with a consistent FDR, as described in detail in Section VI.

In some embodiments, a statistical significance of the tumor burden is determined based on the probability value and a variable significance value. The variable significance value may be determined based on a number of the queried one or more variants while maintaining a predetermined FDR. In certain embodiments, the predetermined FDR is 5%. In other embodiments, the predetermined FDR is less than or equal to 5%, e.g., 2%. In some embodiments, determining the variable significance value comprises: (i) determining a different significance value for each number of the one or more variants of the plurality of variants that are capable of being queried while maintaining the predetermined FDR, and selecting the significance value for the number of the queried one or more variants from the determined different significance values; or (ii) determining an equation relating the significance value to the number of the queried one or more variants while maintaining a predetermined FDR. In additional or alternative embodiments, the significance value is determined based on the number of the queried one or more variants that are associated with the subject tumor.

For example, different p-value cutoffs or significance values may be determined for each number of reporters that are capable of being queried in the sequence data for the sample of cell free DNA, such that the FDR is 5% (or any other chosen FDR) for any number of reporters. This can either be done individually for each number of reporters, or an equation relating the p-value cutoff or significance value to the number of reporters can be determined. The equation relating the p-value cutoff or significance value to the number of reporters queried and the number of non-zero reporters can be determined, such that the FDR is consistently 5% (or any other chosen FDR). The different p-value cutoffs or significance values can be determined by dividing p-value cutoffs or significance values by the number of reporters queried that are associated with a subject tumor (e.g., a disease area or tumor from the subject), then a cutoff can be set for each number of reporters such that the FDR is consistently 5% (or any other chosen FDR).

### III. Sequencing Samples and Analysis System

FIG. 2 shows an example sequence analytical system 200 used in accordance with various embodiments that includes a sample 205, such as a blood sample comprising cfDNA, within a sample holder 210, e.g., a flow cell or a tube containing droplets of cfDNA. A physical characteristic 215, such as a fluorescence intensity value, from the sample 205 is detected by a detector 220. A data signal 225 from the detector 220 can be sent to a data processing system 230 (onboard or separate from the detector), which may include a processor 250 and a memory 235. Data signal 225 may be stored locally in the data processing system 230 in memory 235, or externally in an external memory 240 or a storage device 245. Detector 220 can detect a variety of physical signals, such as light (e.g., fluorescent light from different probes for different bases) or electrical signals (e.g., as created from a molecule traveling through a nanopore). The data processing system 230 may be, or may include, a computer system, ASIC, microprocessor, etc., as described in further detail with respect to FIG. 3. The data processing system 230 may also include or be coupled with a display (e.g., monitor, LED display, etc.) and a user input device (e.g., mouse, keyboard, buttons, etc.). The data processing system 230 and the other components may be part of a stand-alone or network connected computer system, or they may be directly attached to or incorporated in a thermal cycler device. The data processing system 230 may also include optimization software that executes in processor 250. Based on the sequence data, mutations in one or more reads may be quantified and analyzed to determine a tumor burden and a statistical significance of a tumor burden.

An accurate quantification of the mutations requires discriminating PCR duplicate reads from identical molecules that are of unique origin. Computationally, PCR duplicates are identified as sequence reads that align to the same genomic coordinates using reference genome guided alignment. However, identical molecules can be independently generated during library preparation and can have unique cellular origins. Thus, false identification of these molecules as PCR duplicates can lead to erroneous analysis and interpretation of the sequence data. In variant calling methods, sequencing errors may make it further difficult to distinguish actual variant calls from the sequencing artifacts. This problem is more prominent during the detection of low frequency somatic variations, which are expected to be called in liquid biopsy samples, wherein the proportion of ctDNA is very low as compared to normal cfDNA. In order to overcome these challenges, various embodiments include the assignment of unique molecular identifiers to nucleotide molecules while preparing sequencing libraries.

Any of the computer systems or data processing systems described herein may utilize any suitable number of subsystems. An example of a computer system or data processing system (e.g., the data processing system 230 described with respect to FIG. 2) and associate subsystems is shown in FIG. 3. The computing system 300 is only one example of a suitable computing system and is not intended to suggest any limitation as to the scope of use or functionality of the present embodiments. Also, computing system 300 should not be interpreted as having any dependency or requirement relating to any one or combination of components illustrated in sequence analytical system 200.

As shown in FIG. 3, computing system 300 includes a computing device 305. The computing device 305 can be resident on a network infrastructure such as within a cloud environment, or may be a separate independent computing device (e.g., a computing device of a service provider). The computing device 305 may include a bus 310, processor 315, a storage device 320, a system memory (hardware device) 325, one or more input devices 330, one or more output devices 335, and a communication interface 340.

The bus 310 permits communication among the components of computing device 305. For example, bus 310 may be any of several types of bus structures including a memory bus or memory controller, a peripheral bus, and a local bus using any of a variety of bus architectures to provide one or more wired or wireless communication links or paths for transferring data and/or power to, from, or between various other components of computing device 305.

The processor 315 may be one or more conventional processors, microprocessors, or specialized dedicated processors that include processing circuitry operative to interpret and execute computer readable program instructions, such as program instructions for controlling the operation and performance of one or more of the various other components of computing device 305 for implementing the functionality, steps, and/or performance of the present invention. In certain embodiments, processor 315 interprets and executes the processes, steps, functions, and/or operations of the present invention, which may be operatively implemented by the computer readable program instructions. For example, processor 315 can retrieve, e.g., import and/or otherwise obtain or generate sequence data, query the sequence data, calculate mutant molecule loads, model background, determine probability values and significance values, determine tumor burdens, and provide predications such as statistical significance of derived data, interpretive diagnosis, and clinical outcomes. In embodiments, the information obtained or generated by the processor 315, e.g., the sequence data, the mutant molecule loads, background models, probability values and significance values, etc., can be stored in the storage device 320.

The storage device 320 may include removable/non-removable, volatile/non-volatile computer readable media, such as, but not limited to, non-transitory machine readable storage medium such as magnetic and/or optical recording media and their corresponding drives. The drives and their associated computer readable media provide for storage of computer readable program instructions, data structures, program modules and other data for operation of computing device 305 in accordance with the different aspects of the present invention. In embodiments, storage device 320 may store operating system 345, application programs 350, and program data 355 in accordance with aspects of the present invention.

The system memory 325 may include one or more storage mediums, including for example, non-transitory machine readable storage medium such as flash memory, permanent memory such as read-only memory (“ROM”), semi-permanent memory such as random access memory (“RAM”), any other suitable type of non-transitory storage component, or any combination thereof. In some embodiments, an input/output system 360 (BIOS) including the basic routines that help to transfer information between the various other components of computing device 305, such as during start-up, may be stored in the ROM. Additionally, data and/or program modules 365, such as at least a portion of operating system 345, program modules, application programs 350, and/or program data 355, that are accessible to and/or presently being operated on by processor 315, may be contained in the RAM. In embodiments, the program modules 365 and/or application programs 350 can comprise an index or table of know variants or reporters, algorithms or models such as a Monte Carlo algorithm to model background, and a comparison tool, which provides the instructions for execution of processor 315.

The one or more input devices 330 may include one or more mechanisms that permit an operator to input information to computing device 305, such as, but not limited to, a touch pad, dial, click wheel, scroll wheel, touch screen, one or more buttons (e.g., a keyboard), mouse, game controller, track ball, microphone, camera, proximity sensor, light detector, motion sensors, biometric sensor, and combinations thereof. The one or more output devices 335 may include one or more mechanisms that output information to an operator, such as, but not limited to, audio speakers, headphones, audio line-outs, visual displays, antennas, infrared ports, tactile feedback, printers, or combinations thereof.

The communication interface 340 may include any transceiver-like mechanism (e.g., a network interface, a network adapter, a modem, or combinations thereof) that enables computing device 305 to communicate with remote devices or systems, such as a mobile device or other computing devices such as, for example, a server in a networked environment, e.g., cloud environment. For example, computing device 305 may be connected to remote devices or systems via one or more local area networks (LAN) and/or one or more wide area networks (WAN) using communication interface 340.

As discussed herein, computing system 300 may be configured for ultrasensitive detection of circulating nucleic acid with prior knowledge of variants to be monitored in the blood. In particular, computing device 305 may perform tasks (e.g., process, steps, methods and/or functionality) in response to processor 315 executing program instructions contained in non-transitory machine readable storage medium, such as system memory 325. The program instructions may be read into system memory 325 from another computer readable medium (e.g., non-transitory machine readable storage medium), such as data storage device 320, or from another device via the communication interface 340 or server within or outside of a cloud environment. In embodiments, an operator may interact with computing device 305 via the one or more input devices 330 and/or the one or more output devices 335 to facilitate performance of the tasks and/or realize the end results of such tasks in accordance with aspects of the present invention. In additional or alternative embodiments, hardwired circuitry may be used in place of or in combination with the program instructions to implement the tasks, e.g., steps, methods and/or functionality, consistent with the different aspects of the present invention. Thus, the steps, methods and/or functionality disclosed herein can be implemented in any combination of hardware circuitry and software.

IV. Quantification of ctDNA in a Sample Using Mutant Molecules as Defined By the Sequencing Assay, Mutant Molecule Load (MML)

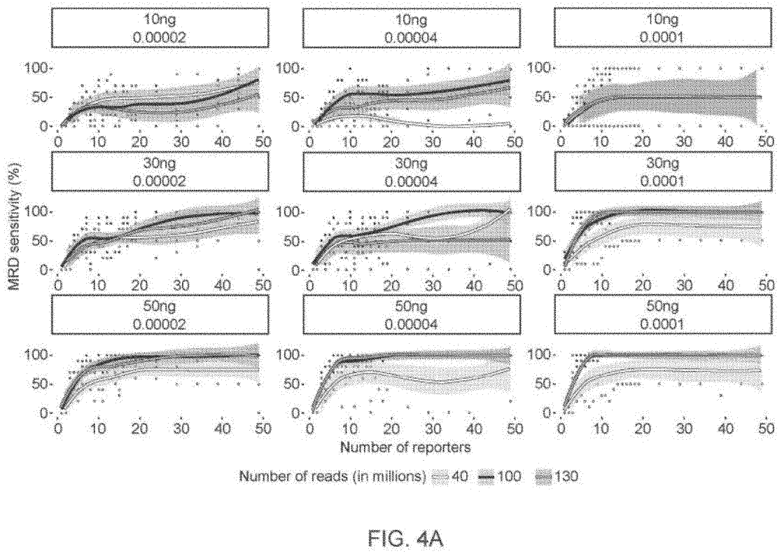

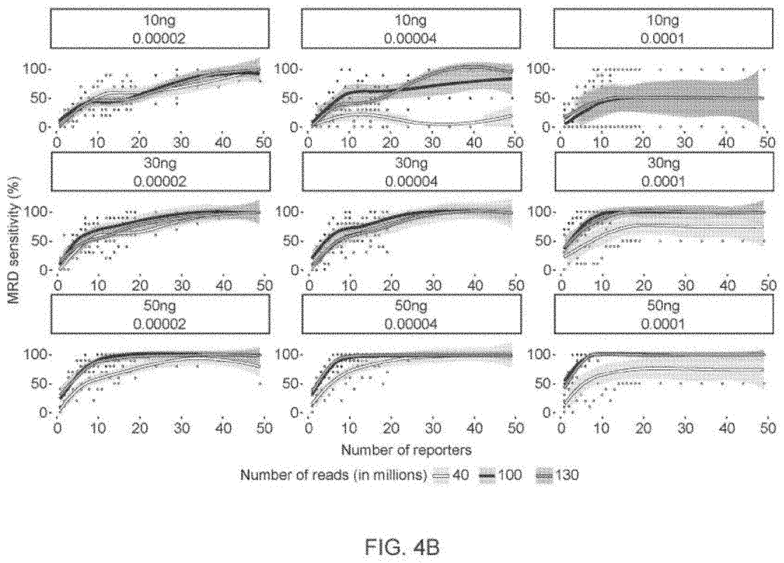

Methods of quantifying ctDNA in plasma typically use either mean allele fraction (AF) of the variants in the plasma, or use the mass of cell free DNA per mL of plasma combined with the mean AF of known variants to calculate the mutant molecules (or mutant copies of the genome) per mL of plasma. However, because the techniques used herein for sequencing the sample of cfDNA may include the use of unique molecule identifiers, the number of sequenced molecules or reads that contain the queried one or more variants can be directly counted (rather than inferred). Consequently, in place of mean AF, or a count of mutant copies of the genome based on mean AF, an actual count of the number of mutant molecules per mL of plasma (first mutant molecule load (MML)) can be calculated and reported. In various embodiments, the unique molecular identifiers (UMIs) are used to properly count input molecules into the assay, the sum or average of mutant molecules across different variants is then combined. The combination of molecule counts from different variants that is unconventional allows for the reporting of mutant genome copies without using the mean AF and adjusting based on ng/mL of plasma (mutant genome copies=sum mutant molecules/number of reporters identified). FIGS. 4A and 4B demonstrate the superior sensitivity observed when using MML as opposed to using mean AF. Specifically, FIG. 4A shows monitoring sensitivity at 0.002%, 0.004% and 0.01% using mean AF, whereas FIG. 4B shows monitoring sensitivity at 0.002%, 0.004% and 0.01% using MML. As shown, the sensitivity of monitoring using MML is higher than the sensitivity of monitoring using mean AF as a measure of tumor burden. In these examples, all values utilized p-value thresholds that correspond to 5% FDR or 95% specificity.

V. Use of Trinucleotide Context of Base Changes from Randomly Selected Positions to Define an Empirically Derived P-Value

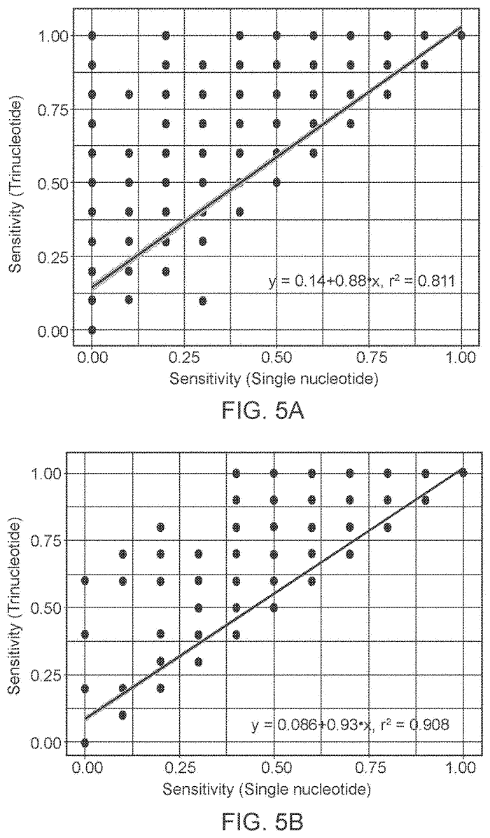

Methods using a Monte Carlo algorithm to model the background noise in a given sample, randomly sample positions in the sequence data from that sample. The random sampling is typically done such that base changes that correspond to the base changes in the list of prior known variants to be monitored are randomly selected (e.g., if a reporter list has 3 variants: A>C, A>T, and G>A, each random sampling would select one A and count the number of molecules supporting a A>C change, one A and count the number of molecules supporting a A>T change, and one G and count the number of molecules supporting a G>A change). While this gives a respectable representation of the background distribution of relevant error types in a given sample, recent data suggests that errors and error rate in querying variants is strongly influenced by the surrounding bases. With this in mind, various embodiments include the random sampling of the background of a sample to account for the context of the surrounding bases. For example, if a reporter list has 3 variants: GAT>GCT, CAC>CTC, and AGT>AAT, each random sampling would select one GAT and count the number of molecules supporting a change to GCT, one CAC and count the number of molecules supporting a change to CTC, and one ATG and count the number of molecules supporting a change to AAT. While the exemplary analyses shown here is based on the trinucleotide context of a variant, it would also be possible to use the dinucleotide, tetranucleotide, pentanucleotide, or other contexts. FIGS. 5A and 5B demonstrate the superior sensitivity observed when using a multinucleotide context as opposed to using a single base change for sampling. Specifically, FIG. 5A shows monitoring sensitivity for 5% FDR using trinucleotides (y-axis) vs. single nucleotides (x-axis) in a first spiked sampled, and FIG. 5B shows monitoring sensitivity for 5% FDR using trinucleotides (y-axis) vs. single nucleotides (x-axis) in a second spiked sampled. As shown, the sensitivity of monitoring using multinucleotide context is higher than the sensitivity of monitoring using single nucleotides as a measure of tumor burden. In these examples, all values utilized MML as measure of mutant molecules.

### VI. Techniques to Ensure Specificity Levels in a Data Derived Manner Using Data-Driven P-Value Threshold Selection

As discussed herein, the p-value cutoff of 0.05 corresponds to the probability of roughly 5% of trials having a second number of mutant molecules (e.g., mean AF or MML) of the background greater than the first number of mutant molecules (e.g., mean AF or MML) derived from the true reporter list, or a false discovery rate (FDR) of 5%. It has recently been discovered that the number of reporters can play a role in determining the exact p-value cutoff for a 5% FDR and the disease area in somatic oncology has implications as well. Accordingly, various aspects concern techniques for the determination of variable p-value cutoffs, which allow detection of ctDNA-positivity with a consistent FDR.

The following example describes a use case and analysis techniques outlined to assess whether the number of reporters and disease area can play a role in determining the exact p-value cutoff for a 5% FDR. The specific use case and analysis techniques are intended to be illustrative rather than limiting.

Initially, reporter lists of single nucleotide variants (SNVs) were generated using samples from The Cancer Genome Atlas (TCGA) separately for non-small cell lung cancer (NSCLC) and colorectal cancer (CRC). Monitoring was performed on a set of known negative samples (healthy donor plasma samples) and p-values were collected for monitoring runs that resulted in a mean AF greater than zero. For each sample, between 3 and 100 reporters were randomly selected, 5 times for each number between 3 and 100. This means that, for each number of reporters, there were 135 observations (27 samples were each queried 5 times). Since the samples, where monitoring was performed, were healthy normal, the expected mean AF measure for tumor burden is zero and any non-zero value is a false positive ctDNA detection event. Based on these results, p-value cutoffs could be modified in the following ways: (1) different p-value cutoffs were determined for each number of reporters, such that the false discovery rate is 5% (or any other value) for any number of reporters. This can either be done individually for each number of reporters, or an equation relating the p-value cutoff to the number of reporters can be determined. (2) An equation relating the p-value cutoff to the number of reporters queried and the number of non-zero reporters can be determined, such that the false discovery rate is consistently 5% (or any other value). (3) Modified p-value cutoffs can be determined by dividing the p-value cutoff by the number of reporters queried, then a cutoff can be set for each number of reporters such that the false discovery rate is consistently 5% (or any other value).

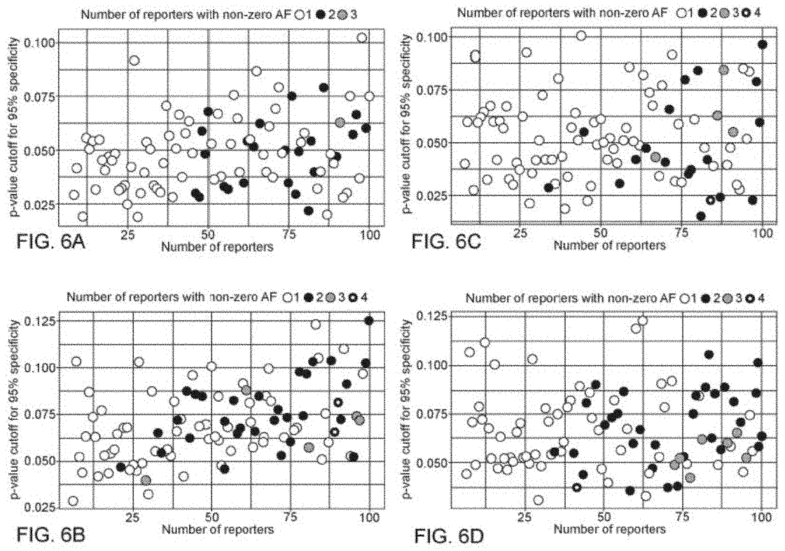

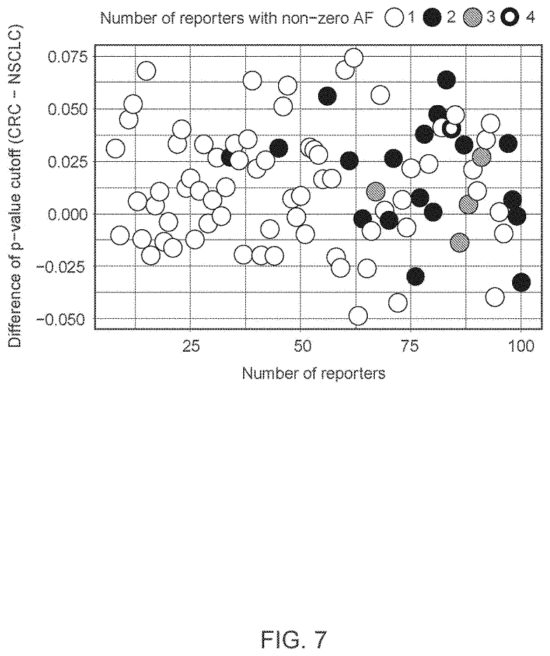

FIGS. 6A-6D illustrate p-value cutoffs for two quantifications of tumor burden, mean AF and MML for NSCLC and CRC. The p-value cutoffs for consistent 5% FDR or 95% specificity are plotted against the number of reporters. Specifically, FIG. 6A shows p-value cutoffs for 5% FDR in NSCLC using mean AF as a measure of tumor burden, FIG. 6B shows p-value cutoffs for 5% FDR in CRC using mean AF as a measure of tumor burden, FIG. 6C shows p-value cutoffs for 5% FDR in NSCLC using MML as a measure of tumor burden, and FIG. 6D shows p-value cutoffs for 5% FDR in CRC using MML as a measure of tumor burden. It should be understood that the p-value cutoffs are different especially depending on the disease area (e.g., NSCLC vs. CRC). The p-value cutoffs for CRC are more permissive than cutoffs for NSCLC. For example, FIG. 7 shows the difference in p-value thresholds for 5% FDR between NSCLC and CRC. For the other studies described herein, p-value thresholds derived from the data behind FIGS. 6A-6D were utilized.

### VII. Methods and Experiment Design

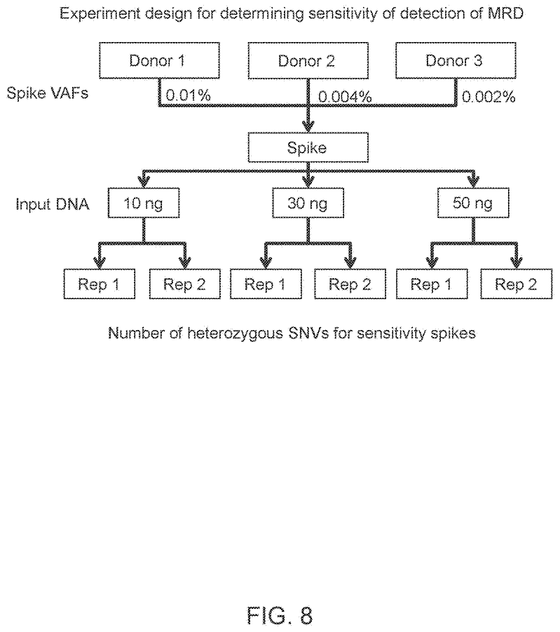

FIG. 8 summarizes the experiment design for the sensitivity experiments described herein. The sensitivity experiments used blends of healthy donor cfDNA samples to create variants at known AFs. cfDNA samples were spiked into each other such that the heterozygous SNPs of three of the samples were at AFs of 0.01%, 0.004%, and 0.002%. These SNPs were treated as reporters for the sensitivity experiments. These spikes were made with two distinct sets of samples (making a first spiked sample and a second spiked sample). Each spiked sample was run in duplicate at 10 ng, 30 ng, and 50 ng of input, and in analysis, each run was subsampled five times to each of 40 million, 100 million, and 130 million reads. For each analysis run, between 1 and 80 reporters were randomly sampled 10 times, and monitoring was performed, considering sum or count of mutant molecule load (MML) and mean AF, performing Monte Carlo algorithms using single nucleotide and trinucleotide context, and P-value cutoffs were set to have a 5% false discovery rate.

### VIII. Diagnostic Assay and Treatment

In various embodiments, techniques are provided for determining whether a subject has minimal residual disease based on the statistical significance of the tumor burden as identified by techniques disclosed herein. Some embodiments further encompass techniques for predicting a clinical outcome of a treatment regimen for the subject or providing a prognosis of cancer in the subject based on the determination of minimal residual disease. For example, once the one or more known variants are identified in the cfDNA and the statistical significance of the tumor burden measured from the one or more known variants is determined, the statistical significance of the tumor burden can be used to determine the presence of minimal residual disease in the subject.

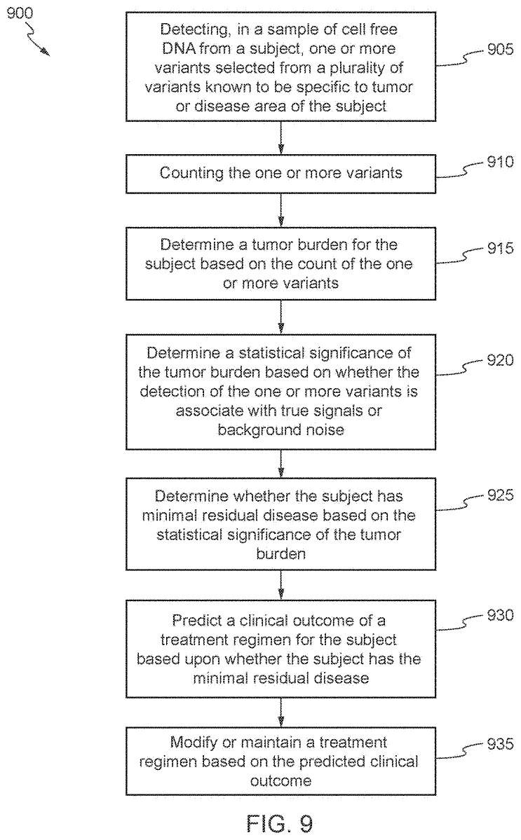

FIG. 9 shows a flowchart 900 that illustrates processes and operations for diagnosing a patient with minimal residual disease. Individual embodiments may be described as a process which is depicted as a flowchart, a flow diagram, a data flow diagram, a structure diagram, or a block diagram, as previously described with respect to FIG. 1. The processes depicted in flowchart 900 include some or all of the steps performed in flowchart 100 described with respect to FIG. 1 and may be implemented by the architecture, systems, and techniques depicted in FIGS. 2 and 3. At step 905, one or more variants selected from a plurality of variants known to be specific to a tumor or disease area of a subject are detected in a sample of cell free DNA from the subject. In some embodiments, the one or more variants are detected using a diagnostic assay. The assay can be created in a variety of ways and use various techniques, such as PCR, sequencing, hybridization arrays, and unique molecule identifiers. The assay should be able to detect ctDNA at least 30× lower than pre-treatment levels. In some embodiments, the assay can be created as part of a kit that comprises reagents necessary for detecting the one or more known variants in the cfDNA. For example, the kit may comprise oligonucleotides such as probes and amplification primers specific for a plurality of variants known to be specific to tumor or disease area of the subject or a population. In some embodiments, the kit further comprises reagents necessary for the performance of amplification and detection assay, such as the components of PCR, a real-time PCR, or transcription mediated amplification (TMA). In some embodiments, the variant-specific oligonucleotides are detectably labeled. In such embodiments, the kit comprises reagents for labeling and detecting the label. For example, if the oligonucleotides are labeled with biotin, the kit may comprise a streptavidin reagent with an enzyme and its chromogenic substrate.

At step 910, the detected one or more variants are counted. In some embodiments, the detected one or more variants are counted using mutant molecules as defined by the assay. For example, because the assay used includes unique molecule identifiers, the number of sequenced molecules that contained the one or more variants may be counted as a mutant molecule load (MML). At step 915, a tumor burden is determined for the subject based on the count of the one or more variants. At step 920, a statistical significance of the tumor burden is determined based on whether the detection of the one or more variants is associate with true signals or background noise. In some embodiments, the statistical significance is determined in accordance with steps 120-140 of flowchart 100 described with respect to FIG. 1. In some embodiments, the statistical significance is determined by: (i) modeling background noise using a multinucleotide context of base changes from randomly selected positions in sequence data for the sample of cell free DNA to define an empirically derived probability value, (ii) determining a variable significance value based on a number of the detected one or more variants while maintaining a predetermined false discovery rate, and (iii) comparing the probability value to the variable significance value, where the lower the probability value compared to the variable significance value, the higher the statistical significance associated with the tumor burden computed from the count of the one or more variants (e.g., if the probability value is less than the variable significance value, then the tumor burden may be determined to be statistically significant).

At step 925, whether the subject has minimal residual disease is determined based on the statistical significance of the tumor burden. The minimal residual disease is the presence of residual tumor that remains in the subject during treatment or after treatment. For example, if the tumor burden is greater than a threshold value and the probability value is less than the significance value, then it may be determined that the subject has minimal residual disease. The threshold value is a predetermined cutoff of a tumor burden value that is associated with the presence of minimal residual disease. Alternatively, if the tumor burden is greater than the threshold value and the probability value is greater than the significance value, then it may be determined that the subject does not have minimal residual disease. Alternatively, if the tumor burden is less than the threshold value and the probability value is less than the significance value, then it may be determined that the subject does not have minimal residual disease. Alternatively, if the tumor burden is less than the threshold value and the probability value is greater than the significance value, then it may be determined that the subject does not have minimal residual disease.

At step 930, a clinical outcome of a treatment regimen for the subject is predicted based upon whether the subject has the minimal residual disease. Several studies have confirmed the importance of assessing the potential presence of minimal residual disease during or following a treatment regimen, to aid in predicting clinical outcomes of patients. For example, patients who do not exhibit sustained minimal residual disease, fare significantly better than patients who exhibit sustained minimal residual disease. At step 935, upon determining the subject does have minimal residual disease and predicting a negative clinical outcome, the treatment regimen of the subject may be modified. Alternatively, upon determining the subject does not have minimal residual disease and predicting a positive clinical outcome, the treatment regimen of the subject may be maintained.

IX. Early Assessment of Therapy Response via Longitudinal ctDNA Analysis Including Mutant DNA Molecule Counting

Despite routine use of chemoradiation therapy in cancer patients, our knowledge of optimal prognostic methods is still limited to imaging-based methods. However, it has now been discovered that an early response assessed by the level of ctDNA in a subject (e.g., a determination of whether a subject has minimal residual disease) after starting therapy could predict treatment effect. In various embodiments, techniques are provided for performing a survivability analysis based on the use of known variants for monitoring ctDNA levels, as identified by techniques disclosed herein; where if a subject is classified as a responder “early in treatment” based on ctDNA levels, then it is predicted the subject will have a better treatment effect as opposed to a non-responder.

The following examples describe various use cases and analysis techniques outlined to assess whether there is an association between ctDNA levels and treatment effect. The specific use cases and analysis techniques are intended to be illustrative rather than limiting.

### EXAMPLE 1

Experimental methods to determine whether there is an association between ctDNA levels and treatment effect included the use of a 197-gene next generation sequencing (NGS) assay, which allowed for the performance of a longitudinal ctDNA analysis for multiple subjects in an advanced NSCLC Study. As used herein, a “longitudinal study” is an observational research method in which data is gathered for the same subjects repeatedly over a period of time and the mutant molecules per milliliter-of-plasma (MMPM) or mutant molecule load (MML) is measure based on the data, which quantifies ctDNA at the variant level. With variants identified in plasma before the start of current treatment of each subject, it was possible to monitor disease burden after the first and second cycles of therapy using the techniques disclosed herein. The association between ctDNA levels and survival was evaluated in a cohort of advanced lung adenocarcinoma subjects. Post-treatment MMPM or MML values were compared with the MMPM or MML value at baseline and/or a previous treatment time point.

In particular, at baseline (b0), variants were identified in all subjects to enable ctDNA monitoring. The plasma draws after the first and second treatment cycle were defined as p1 and p2, respectively. By applying a Continuous Responder algorithm, it was possible to classify each subject as either a “responder” or a “non-responder”. The characterization of this binary outcome is often based on a dichotomy of a continuous outcome variable and the classification is intended to capture a magnitude of effect that is deemed to be clinically relevant. The Continuous Responder algorithm was defined by a continuous drop in ctDNA levels represented by mean MMPM or MML reduction over time (p2<p1<b0), and was applied to a mean MMPM or MML below 8 at p2. As a result, continuous responders 13/43 (30%) were associated with a better therapy response indicated by a progression free survival (PFS) curve (P=0.028; HR 0.45; 95% CI 0.23-0.90) and overall survival (OS) curve (P=0.0074; HR 0.3; 95% CI 0.12-0.77). The continuous responders demonstrated a median overall survival benefit of 11.25 months over the poor responders. Thus, early post-treatment ctDNA level measured by NGS and the techniques described herein are associated with chemoradiation therapy response in advanced lung adenocarcinoma subjects.

### EXAMPLE 2

Experimental methods to determine whether there is an association between ctDNA levels and treatment effect included the use of a 197-gene next generation sequencing (NGS) assay, which allowed for the performance of a longitudinal ctDNA analysis for multiple subjects in a German Lung Cancer Multi-Marker Study. Data was gathered for the same subjects repeatedly over a period of time and the mutant molecules per milliliter-of-plasma (MMPM) or mutant molecule load (MML) was measured based on the data, which quantifies the ctDNA at the variant level. All extracted cfDNA samples were processed and sequenced in order of date of blood draw. With variants identified in plasma before the start of current treatment of each subject, it was possible to monitor disease burden after the first and second cycles of therapy using the techniques disclosed herein. The association between ctDNA levels and survival was evaluated in 72 consecutive small-cell lung cancer (SCLC) patients, UICC-stage IIIB/IV, where plasma samples were available prior to start of therapy, and prior to the second cycle of chemotherapy, and prior to the third cycle of chemotherapy. Post-treatment MMPM or MML values were compared with the MMPM or MML value at baseline and/or a previous treatment time point.

In particular, at baseline (b0), variants were identified in all (72/72) subjects to enable ctDNA monitoring. Using serial liquid biopsies from each subject, the mean MMPM or MML at post-first treatment cycle (p1) and the mean MMPM or MML at post-second treatment cycle (p2) were analyzed. By applying a Continuous Responder algorithm, it was possible to classify each subject as either a “responder” or a “non-responder”. The Continuous Responder algorithm was defined by a continuous drop in ctDNA levels represented by mean MMPM or MML reduction over time (p2<p1<b0), to a mean MMPM below 18 at p2. As a result, continuous responders 47/72 were associated with a better therapy response OS curve (HR=2; P=0.0092%; CI 1.2-3.4). The continuous responders demonstrated a median survival benefit of 4.6 months over the poor responders. Neither gender, nor age, nor eastern cooperative oncology group performance status, nor stage were predictors of response in the models. Thus, early post-treatment ctDNA level measured by NGS and the techniques described herein are associated with chemoradiation therapy response in advanced SCLC subjects.

### Techniques for Performing a Survivability Analysis

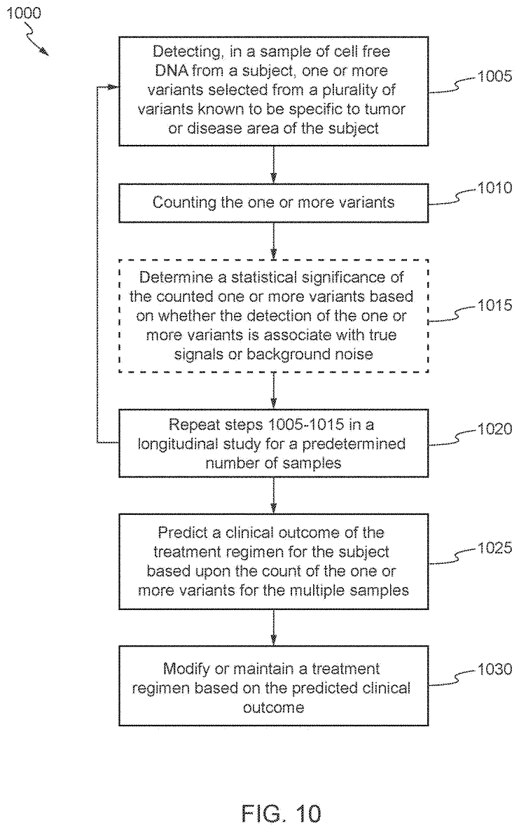

FIG. 10 shows a flowchart 1000 that illustrates processes and operations for performing a survivability analysis. Individual embodiments may be described as a process which is depicted as a flowchart, a flow diagram, a data flow diagram, a structure diagram, or a block diagram, as previously described with respect to FIG. 1. The processes depicted in flowchart 1000 include some or all of the steps performed in flowchart 100 described with respect to FIG. 1 and may be implemented by the architecture, systems, and techniques depicted in FIGS. 2 and 3. At step 1005, one or more variants selected from a plurality of variants known to be specific to tumor or disease area of a subject are detected in a sample of cell free DNA from the subject. In some embodiments, the sample of cell free DNA is obtained from the subject prior to the start of a treatment regimen. In some embodiments, the one or more variants are detected using a diagnostic assay, as described herein. At step 1010, the detected one or more variants are counted. In some embodiments, the detected one or more variants are counted by measuring the mutant molecules per milliliter-of-plasma (MMPM). In other embodiments, the detected one or more variants are counted using mutant molecules as defined by the assay. For example, because the assay used includes unique molecule identifiers, the number of sequenced molecules that contained the one or more variants may be counted as a mutant molecule load (MML). In other embodiments, the count of the one or more variants is used to calculate a tumor burden, as described herein. Optionally, at step 1015, a statistical significance of the count of the determined one or more variants (or quantitation of the tumor burden (either MML or mean AF)) is determined based on whether the detection of the one or more variants is associate with true signals or background noise. In some embodiments, the statistical significance is determined in accordance with steps 120-140 of flowchart 100 described with respect to FIG. 1. In some embodiments, the statistical significance is determined by: (i) modeling the background noise using a multinucleotide context of base changes from randomly selected positions in sequence data for the cell free DNA to define an empirically derived probability value, (ii) determining a variable significance value based on a number of the detected one or more variants (or quantitation of the tumor burden (either MML or mean AF)) while maintaining a predetermined false discovery rate, and (iii) comparing the probability value to the variable significance value, where the lower the probability value compared to the variable significance value, the higher the statistical significance associated with the count of the one or more variants (e.g., if the probability value is less than the variable significance value, then the count of the determined one or more variants may be determined to be statistically significant).

At step 1020, steps 1005-1015 are repeated for a predetermined number of samples of cell free DNA from the subject at various points prior to and during a treatment regimen to obtain the count of the one or more variants for each of the samples of cell free DNA and the statistical significance associated with each of the counts of the one or more variants. For example, a longitudinal study is performed for the subject using analysis of one or more variants in multiple samples of cell free DNA. In some embodiments, the steps 1005-1015 are repeated pre-treatment regimen and prior to or after early cycles in the treatment regimen. In some embodiments, the steps 1005-1015 are repeated pre-treatment regimen and prior to or after cycles through-out the treatment regimen. In some embodiments, the steps 1005-1015 are repeated pre-treatment regimen, prior to or after cycles during the treatment regimen, and post-treatment regimen.

At step 1025, a clinical outcome of the treatment regimen for the subject is predicted based upon the count of the one or more variants for each of the samples of cell free DNA and the statistical significance associated with each of the counts of the one or more variant. The study discussed herein has confirmed the importance of assessing the potential presence of ctDNA during a treatment regimen, to aid in predicting clinical outcomes of patients. For example, patients who demonstrate a continuous drop in ctDNA levels represented by mean MMPM or MML reduction over time (p2<p1<b0), fare significantly better than patients who fail to exhibit a continuous drop in ctDNA levels. In some embodiments, the count of the one or more variants for each of the samples of cell free DNA and the statistical significance associated with each of the counts of the one or more variant are analyzed using a continuous responder algorithm to classify the subject as a responder or a non-responder in order to predict the clinical outcome of the treatment regimen for the subject. When the subject is classified as a responder, the clinical outcome of the treatment regimen for the subject is predicted to be a positive clinical outcome. When the subject is classified as a non-responder, the clinical outcome of the treatment regimen for the subject is predicted to be a negative clinical outcome.

At step 1030, upon predicting a negative clinical outcome, the treatment regimen of the subject may be modified. Alternatively, upon predicting a positive clinical outcome, the treatment regimen of the subject may be maintained.

1. A method comprising:
(a) obtaining, by a data processing system, sequence data for a plurality of target regions in a sample of cell free DNA from a subject, wherein the plurality of target regions are selected from a plurality of genomic regions that comprise a plurality of known variants, and the plurality of known variants are tagged with unique molecular identifiers;
(b) querying, by the data processing system, the sequence data for one or more variants of the plurality of known variants;
(c) calculating, by the data processing system, a first mutant molecule load (MML) for each of the queried one or more variants based on a first count of the unique molecular identifiers for each of the queried one or more variants;
(d) modeling, by the data processing system, background in the sample of cell free DNA, wherein the modeling comprises randomly sampling the sequence data a predetermined number of times for one or more types of variants present in the plurality of genomic regions and calculating a second MML for each of the one or more variants in the random samples based on a second count of the unique molecular identifiers for each of the one or more variants in the random samples;
(e) comparing, by the data processing system, the second MML for each of the one or more variants in the random samples to the first MML for each of the queried one or more variants;
(f) generating, by the data processing system, a ratio based on the comparison, wherein the ratio is: (a number of the random samples where the second MML is greater than the first MML):(the predetermined number of times), and the ratio is a probability value for a null-hypothesis that the second MML of the background is greater than the first MML of the queried variant;
(g) determining, by the data processing system, a tumor burden for the subject based on the first MML for each of the queried one or more variants; and
(h) determining, by the data processing system, a statistical significance of the tumor burden based on the probability value and a significance value.

2. The method of claim 1, wherein the modeling further comprises determining, by the data processing system, a distribution of the one or more types of variants present in the plurality of genomic regions, wherein the distribution comprises a first type of variant and a second type of variant.

3. The method of claim 2, wherein the randomly sampling the sequence data comprises: (i) selecting at least one base associated with the first type of variant a predetermined number of times from the sequence data and counting a number of molecules supporting the first type of variant, and (ii) selecting at least one base associated with the second type of variant a predetermined number of times from the sequence data and counting a number of molecules supporting the second type of variant.

4. The method of claim 3, wherein the at least one base associated with the first type of variant is selected based on a multinucleotide context of the first type of variant, and the at least one base associated with the second type of variant is selected based on the multinucleotide context of the second type of variant.

5. The method of claim 1, further comprising determining, by the data processing system, the significance value as a variable significance value based on a number of the queried one or more variants while maintaining a predetermined false discovery rate.

6. The method of claim 5, wherein the determining the variable significance value comprises: (i) determining a different significance value for each number of the one or more variants of the plurality of variants that are capable of being queried while maintaining the predetermined false discovery rate, and selecting the significance value for the number of the queried one or more variants from the determined different significance values; or (ii) determining an equation relating the significance value to the number of the queried one or more variants while maintaining a predetermined false discovery rate.

7. The method of claim 5, wherein the significance value is determined based on the number of the queried one or more variants that are associated with a subject tumor.

8. The method of claim 1, further comprising determining, by the data processing system, whether the subject has minimal residual disease based on the statistical significance of the tumor burden.

9. The method of claim 8, further comprising:
predicting, by the data processing system, a clinical outcome of a treatment regimen for the subject based upon whether the subject has the minimal residual disease; and
upon determining the subject does have minimal residual disease and predicting a negative clinical outcome, modifying the treatment regimen of the subject.

10. A method comprising:
(a) obtaining, by a data processing system, sequence data for a plurality of target regions in a sample of cell free DNA from a subject, wherein the plurality of target regions are selected from a plurality of genomic regions that comprise a plurality of known variants;
(b) querying, by the data processing system, the sequence data for one or more variants of the plurality of known variants;
(c) calculating, by the data processing system, a first number of mutant molecules for each of the queried one or more variants based on an allele fraction for each of the queried one or more variants;
(d) modeling, by the data processing system, background in the sample of cell free DNA, wherein the modeling comprises randomly sampling the sequence data a predetermined number of times for one or more types of variants present in the plurality of genomic regions and calculating a second number of mutant molecules for each of the one or more variants in the random samples based on an allele fraction for each of the one or more variants in the random samples;
(e) comparing, by the data processing system, the second number of mutant molecules for each of the one or more variants in the random samples to the first number of mutant molecules for each of the queried one or more variants;
(f) generating, by the data processing system, a ratio based on the comparison, wherein the ratio is: (a number of the random samples where the second number of mutant molecules is greater than the first number of mutant molecules):(the predetermined number of times), and the ratio is a probability value for a null-hypothesis that the second number of mutant molecules of the background is greater than the first number of mutant molecules of the queried variant;
(g) determining, by the data processing system, a variable significance value based on a number of the queried one or more variants while maintaining a predetermined false discovery rate, wherein the determining the variable significance value comprises: (i) determining a different significance value for each number of the one or more variants of the plurality of known variants that are capable of being queried while maintaining the predetermined false discovery rate, and selecting the significance value for the number of the queried one or more variants from the determined different significance values; or (ii) determining an equation relating the significance value to the number of the queried one or more variants while maintaining a predetermined false discovery rate;
(h) determining, by the data processing system, a tumor burden for the subject based on the first number of mutant molecules for each of the queried one or more variants; and
(i) determining, by the data processing system, a statistical significance of the tumor burden based on the probability value and the variable significance value.

11. The method of claim 10, the modeling further comprises determining, by the data processing system, a distribution of the one or more types of variants present in the plurality of genomic regions, wherein the distribution comprises a first type of variant and a second type of variant.

12. A method of diagnosing a patient with minimal residual disease comprising:
(a) detecting, by a data processing system, one or more variants in a sample of cell free DNA from a subject, wherein the one or more variants are selected from a plurality of variants known to be specific to a tumor or disease area of the subject;
(b) counting, by the data processing system, the detected one or more variants;
(c) determining, by the data processing system, a tumor burden based on the count of the one or more variants;
(d) determining, by the data processing system, a statistical significance of the tumor burden, wherein the determining the statistical significance comprises: (i) modeling background noise using a multinucleotide context of base changes from randomly selected positions in sequence data for the sample of cell free DNA to define an empirically derived probability value, (ii) determining a variable significance value based on a number of the detected one or more variants while maintaining a predetermined false discovery rate, and (iii) comparing the probability value to the variable significance value, where the lower the probability value compared to the variable significance value, the higher the statistical significance of the tumor burden; and
(e) determining, by the data processing system, whether the subject has minimal residual disease based on the statistical significance of the tumor burden.

13. A method of performing a survivability analysis, the method comprising:
(a) detecting, by a data processing system, one or more variants in a sample of cell free DNA from a subject, wherein the one or more variants are selected from a plurality of variants known to be specific to a tumor or disease area of the subject;
(b) counting, by the data processing system, the detected one or more variants;
(c) determining, by the data processing system, a statistical significance of the count of the one or more variants, wherein the determining the statistical significance comprises: (i) modeling background noise using a multinucleotide context of base changes from randomly selected positions in sequence data for the sample of cell free DNA to define an empirically derived probability value, (ii) determining a variable significance value based on a number of the detected one or more variants while maintaining a predetermined false discovery rate, and (iii) comparing the probability value to the variable significance value, where the lower the probability value compared to the variable significance value, the higher the statistical significance of the count of the one or more variants;
(d) repeating steps (a)-(c) for a predetermined number of samples of cell free DNA from the subject at various points prior to and during a treatment regimen to obtain the count of the one or more variants for each of the samples of cell free DNA and the statistical significance associated with each of the counts of the one or more variants; and
(e) predicting, by the data processing system, a clinical outcome of the treatment regimen for the subject based upon the count of the one or more variants for each of the samples of cell free DNA and the statistical significance associated with each of the counts of the one or more variants.

14. A system comprising:
one or more processors;
a memory accessible to the one or more processors, the memory storing a plurality of instructions executable by the one or more processors, the plurality of instructions comprising instructions that when executed by the one or more processors cause the one or more processors to perform the method of claim 1.

15. A computer product comprising a computer readable medium storing a plurality of instructions for controlling a computer system to perform the method of claim 1.
